(19) United States
(12) Patent Application Publication
(10) Pub. No.: US 2011/0251476 A1
(43) Pub. Date: Oct. 13, 2011

(54) ARRANGEMENT AND METHOD FOR MEASURING THE LOCAL VELOCITY OF A LIQUID

(75) Inventors: Bernhard Gleich, Hamburg (DE); Juergen Weizenecker, Eggenstein (DE)

(73) Assignee: KONINKLIJKE PHILIPS ELECTRONICS N.V., EINDHOVEN (NL)

(21) Appl. No.: 13/133,742

(22) PCT Filed: Dec. 7, 2009
(86) PCT No.: PCT/IB2009/055542

§ 371 Date: Jun. 9, 2011

(30) Foreign Application Priority Data
Dec. 12, 2008 (EP) 08171427.1

(51) Int. Cl. A61B 5/055

(52) U.S. Cl. 600/409

(57) ABSTRACT

The present invention relates to an arrangement (10) and a corresponding method for measuring the local velocity of a liquid containing a magnetic material (100) in a vessel within a region of action (300), which are particularly suitable to measure the local velocity of the blood in the coronary arteries. This is achieved by following fluctuations of the liquid (containing magnetic particles) in a bolus.

### FIELD OF THE INVENTION

The present invention relates to an arrangement and a corresponding method for measuring the local velocity of a liquid containing a magnetic material in a vessel within a region of action. Further, the present invention relates to a computer program.

### BACKGROUND OF THE INVENTION

An arrangement for magnetic particle imaging (MPI) is known from German patent application DE 101 51 778 A1. In the arrangement described in that publication, first of all a magnetic selection field having a spatial distribution of the magnetic field strength is generated such that a first sub-zone having a relatively low magnetic field strength and a second sub-zone having a relatively high magnetic field strength are formed in the examination zone. The position in space of the sub-zones in the examination zone is then shifted, so that the magnetization of the particles in the examination zone changes locally. Signals are recorded which are dependent on the magnetization in the examination zone, which magnetization has been influenced by the shift in the position in space of the sub-zones, and information concerning the spatial distribution of the magnetic particles in the examination zone is extracted from these signals, so that an image of the examination zone can be formed. Such an arrangement has the advantage that it can be used to examine arbitrary examination objects—e.g. human bodies—in a non-destructive manner and without causing any damage and with a high spatial resolution, both close to the surface and remote from the surface of the examination object.

A similar arrangement and method is known from Gleich, B. and Weizenecker, J. (2005), “Tomographic imaging using the nonlinear response of magnetic particles” in nature, vol. 435, pp. 1214-1217. The arrangement and method for magnetic particle imaging described in that publication takes advantage of the non-linear magnetization curve of small magnetic particles.

The cardiac imaging market is very attractive. Especially imaging of coronaries is important. While MPI has enough temporal resolution, the spatial resolution with currently available tracer material is not sufficient to diagnose a stenosis directly.

### SUMMARY OF THE INVENTION

It is an object of the present invention to provide an arrangement and a corresponding method for measuring the local velocity of a liquid containing a magnetic material in a vessel within a region of action, which are particularly suitable to measure the local velocity of the blood in the coronary arteries.

In a first aspect of the present invention an arrangement is presented comprising:
selection means for generating a magnetic selection field having a pattern in space of its magnetic field strength such that a first sub-zone having a low magnetic field strength and a second sub-zone having a higher magnetic field strength are formed in the region of action,
drive means for changing the position in space of the two sub-zones in the region of action by means of a magnetic drive field so that the magnetization of the magnetic material changes locally,
receiving means for acquiring detection signals, which detection signals depend on the magnetization in the region of action, which magnetization is influenced by the change in the position in space of the first and second sub-zone,
control means for controlling the drive means to change the position in space of the first sub-zone so that it follows the vessel and for controlling the receiving means to acquire at least two detection signals at different positions of the first sub-zone along the vessel, and
correlation means for correlating two of the at least two detections signals and for determining from the correlated detection signal and the known distance between the positions of the first sub-zone, at which said detections signals were acquired, the local velocity of the liquid.

In a further aspect of the present invention a corresponding method and a computer program comprising program code means for causing a computer to control an arrangement as claimed in claim 1 to carry out the steps of the method as claimed in claim 9 when said computer program is carried out on the computer are presented.

Preferred embodiments of the invention are defined in the dependent claims. It shall be understood that the claimed method and computer program have similar and/or identical preferred embodiments as the claimed device and as defined in the dependent claims.

The present invention is based on the idea to measure the local velocities of a liquid in a vessel, e.g. of blood in the coronary arteries, by following fluctuations of the liquid (containing magnetic particles) in a bolus.

Basically, in an embodiment, the movement of a concentration fluctuation is tracked using MPI. For this a first bolus is applied and the position of the coronaries is determined for the different heart phases using heart images of the first bolus. After segmenting the coronaries of interest, a focus field sequence is generated following the coronaries with high velocity (e.g. 10 m/s). Along the focus field path, MPI images are recorded using a drive field with the main components ideally along the coronary. Images are reconstructed in real time and the focus field path is corrected to keep the highest intensity approximately in the middle of the field of view.

In a further embodiment the local blood velocity is determined by picking two voxels and maximizing the correlation between the signal in the first voxel and the time shifted signal in the second voxel. The determined time shift together with the distance of the voxels gives the velocity. This is preferably done for all voxel pairs with short enough distance. The resulting velocities may be displayed colour coded on top of the concentration image.

Preferably, said control means is adapted for controlling the receiving means to acquire a plurality of detection signals at different positions of the first sub-zone along the vessel and said correlation means is adapted for manifold correlating different two detection signals of the plurality of detections signals and for manifold determining from the correlated detection signal and the known distance between the positions of the first sub-zone, at which said detections signals were acquired, the local velocity of the liquid at different positions along the vessel. This improves accuracy of the measurement.

Further, it is preferred that said control means is adapted for using a roadmap of the vessel along which the at least two detection signals shall be acquired and for using said roadmap to control the drive means. This roadmap can be acquired in advance, e.g. by a different imaging modality, such as MR or CT, or can be acquired by the MPI arrangement before the measurement of the velocity of the liquid in the vessel is performed.

It is also possible that said control means is adapted for controlling the drive means and the receiving means to acquire detection signals at different positions of the first sub-zone in said region of action while a bolus of a medium, in particular a contrast agent, containing said magnetic material is passing said different positions, and

further comprising a segmentation means for segmenting vessels from said acquired detection signals to obtain said roadmap.

In addition, focus means can be provided for changing the position in space of the region of action by means of a magnetic focus field and the control means can be adapted for controlling the focus means to move the region of action for the acquisition of said roadmap. Such a focus field has the same (or similar) spatial distribution as the drive field. Separate (preferably) or the same means (e.g. coils) can be used as the focus means and the drive means. The basic difference is that the frequencies are much lower (e.g. <1 kHz, typically <100 Hz) for the focus field that for the drive field, but the amplitudes of the focus field are much higher (e.g. 200 mT compared to 20 mT for the drive field). These fields are used to move the FFP to a desired position. The drive field is required in addition to the focus field since the detection signal obtainable with only the focus field would not be usable for the desired purpose, as the frequencies produced in the object of interest a much too low (typically <10 kHz). Preferably, reconstruction means are provided in addition for reconstructing a concentration image of said region of action from said detections signal acquired at different positions of the first sub-zone in said region of action. The measured velocity information can then, for instance, be indicated in such a concentration image.

According to another embodiment said control means is adapted for controlling the drive means and/or the focus means to change the position in space of the first sub-zone such that it substantially moves in a direction along the expected movement of the liquid in the vessel. This has the advantage that the vessel needs to followed with a much less accuracy since the sensitive area transverse to the direction of movement is relatively broad.

According to still another embodiment said control means is adapted for controlling the drive means and/or the focus means to change the position in space of the first sub-zone such that it substantially moves on a surface of a known object, e.g. the surface of the heart. The direction of movement is then generally in a tangential direction to the surface (e.g. of the heart). This embodiment is particularly useful in case the roadmap, e.g. of the coronaries, is not known, but the surface of the object, e.g. the heart, is well known. Two-dimensional (radial) MPI sequences are preferably used for this purpose.

### BRIEF DESCRIPTION OF THE DRAWINGS

These and other aspects of the invention will be apparent from and elucidated with reference to the embodiment(s) described hereinafter. In the following drawings

FIG. 1 shows a schematic view of the principle layout of a magnetic particle imaging (MPI) arrangement,

FIG. 2 shows an example of the field line pattern produced by an arrangement according to the present invention,

FIG. 3 shows an enlarged view of a magnetic particle present in the region of action,

FIGS. 4a and 4b show the magnetization characteristics of such particles,

FIG. 5 shows a block diagram of the apparatus according to the present invention, and

FIG. 6 shows part of a vessel tree illustrating the present invention.

### DETAILED DESCRIPTION OF THE INVENTION

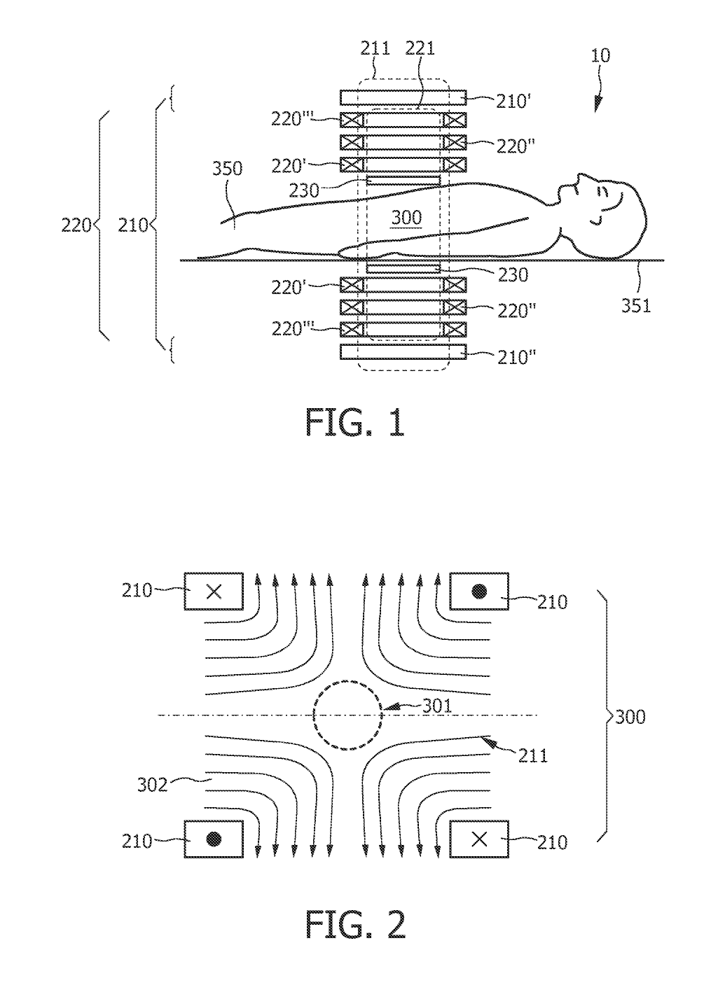

FIG. 1 shows an arbitrary object to be examined by means of a MPI arrangement 10. The reference numeral 350 in FIG. 1 denotes an object, in this case a human or animal patient, who is arranged on a patient table 351, only part of the top of which is shown. Prior to the application of the method according to the present invention, magnetic particles 100 (not shown in FIG. 1) are arranged in a region of action 300 of the inventive arrangement 10. Especially prior to a therapeutical and/or diagnostical treatment of, for example, a tumor, the magnetic particles 100 are positioned in the region of action 300, e.g. by means of a liquid (not shown) comprising the magnetic particles 100 which is injected into the body of the patient 350.

As an example of an embodiment of the present invention, an arrangement 10 is shown in FIG. 2 comprising a plurality of coils forming a selection means 210 whose range defines the region of action 300 which is also called the region of treatment 300. For example, the selection means 210 is arranged above and below the patient 350 or above and below the table top. For example, the selection means 210 comprise a first pair of coils 210′, 210″, each comprising two identically constructed windings 210′ and 210″ which are arranged coaxially above and below the patient 350 and which are traversed by equal currents, especially in opposed directions. The first coil pair 210′, 210″ together are called selection means 210 in the following. Preferably, direct currents are used in this case. The selection means 210 generate a magnetic selection field 211 which is in general a gradient magnetic field which is represented in FIG. 2 by the field lines. It has a substantially constant gradient in the direction of the (e.g. vertical) axis of the coil pair of the selection means 210 and reaches the value zero in a point on this axis. Starting from this field-free point (not individually shown in FIG. 2), the field strength of the magnetic selection field 211 increases in all three spatial directions as the distance increases from the field-free point. In a first sub-zone 301 or region 301 which is denoted by a dashed line around the field-free point the field strength is so small that the magnetization of particles 100 present in that first sub-zone 301 is not saturated, whereas the magnetization of particles 100 present in a second sub-zone 302 (outside the region 301) is in a state of saturation. The field-free point or first sub-zone 301 of the region of action 300 is preferably a spatially coherent area; it may also be a punctiform area or else a line or a flat area. In the second sub-zone 302 (i.e. in the residual part of the region of action 300 outside of the first sub-zone 301) the magnetic field strength is sufficiently strong to keep the particles 100 in a state of saturation. By changing the position of the two sub-zones 301, 302 within the region of action 300, the (overall) magnetization in the region of action 300 changes. By measuring the magnetization in the region of action 300 or a physical parameters influenced by the magnetization, information about the spatial distribution of the magnetic particles in the region of action can be obtained. In order to change the relative spatial position of the two sub-zones 301, 302 in the region of action 300, a further magnetic field, the so-called magnetic drive field 221, is superposed to the selection field 211 in the region of action 300 or at least in a part of the region of action 300.

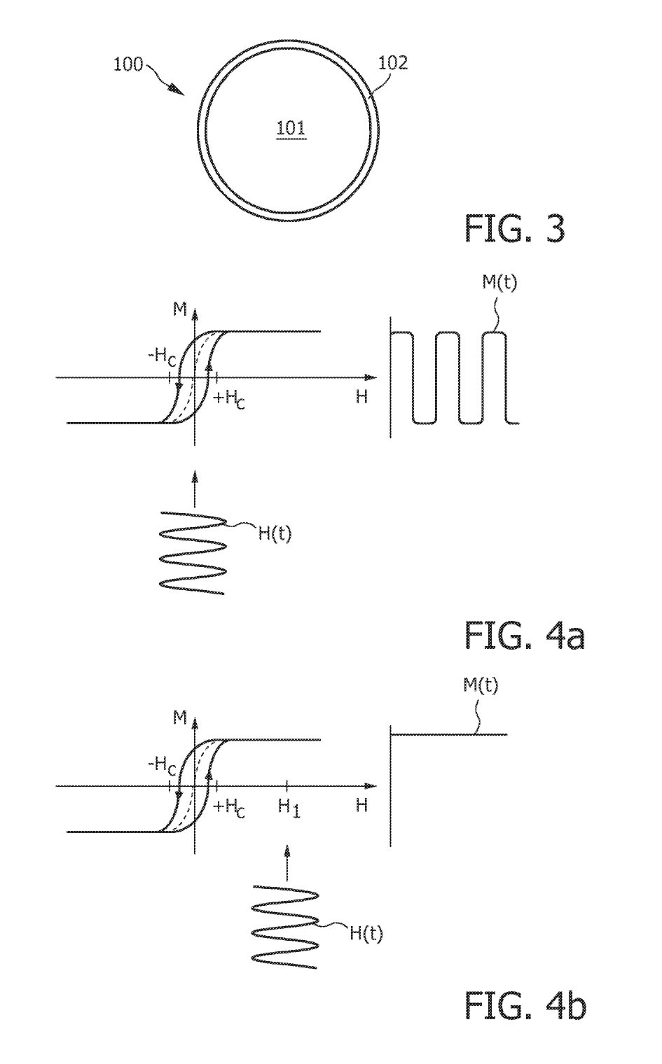

FIG. 3 shows an example of a magnetic particle 100 of the kind used together with an arrangement 10 of the present invention. It comprises for example a spherical substrate 101, for example, of glass which is provided with a soft-magnetic layer 102 which has a thickness of, for example, 5 nm and consists, for example, of an iron-nickel alloy (for example, Permalloy). This layer may be covered, for example, by means of a coating layer 103 which protects the particle 100 against chemically and/or physically aggressive environments, e.g. acids. The magnetic field strength of the magnetic selection field 211 required for the saturation of the magnetization of such particles 100 is dependent on various parameters, e.g. the diameter of the particles 100, the used magnetic material for the magnetic layer 102 and other parameters.

In the case of e.g. a diameter of 10 μm, a magnetic field of approximately 800 A/m (corresponding approximately to a flux density of 1 mT) is then required, whereas in the case of a diameter of 100 μm a magnetic field of 80 A/m suffices. Even smaller values are obtained when a coating 102 of a material having a lower saturation magnetization is chosen or when the thickness of the layer 102 is reduced.

For further details of the preferred magnetic particles 100, the corresponding parts of DE 10151778 are hereby incorporated by reference, especially paragraphs 16 to 20 and paragraphs 57 to 61 of EP 1304542 A2 claiming the priority of DE 10151778.

According to the present invention, in particular for imaging coronary arteries, other magnetic particles can be used as well, e.g. a contrast agent marketed under the name Resovist.

The size of the first sub-zone 301 is dependent on the one hand on the strength of the gradient of the magnetic selection field 211 and on the other hand on the field strength of the magnetic field required for saturation. For a sufficient saturation of the magnetic particles 100 at a magnetic field strength of 80 A/m and a gradient (in a given space direction) of the field strength of the magnetic selection field 211 amounting to 160 10³ A/m2, the first sub-zone 301 in which the magnetization of the particles 100 is not saturated has dimensions of about 1 mm (in the given space direction).

When a further magnetic field—in the following called a magnetic drive field 221 is superposed on the magnetic selection field 210 (or gradient magnetic field 210) in the region of action 300, the first sub-zone 301 is shifted relative to the second sub-zone 302 in the direction of this magnetic drive field 221; the extent of this shift increases as the strength of the magnetic drive field 221 increases. When the superposed magnetic drive field 221 is variable in time, the position of the first sub-zone 301 varies accordingly in time and in space. It is advantageous to receive or to detect signals from the magnetic particles 100 located in the first sub-zone 301 in another frequency band (shifted to higher frequencies) than the frequency band of the magnetic drive field 221 variations. This is possible because frequency components of higher harmonics of the magnetic drive field 221 frequency occur due to a change in magnetization of the magnetic particles 100 in the region of action 300 as a result of the non-linearity of the magnetization characteristics.

In order to generate these magnetic drive fields 221 for any given direction in space, there are provided three further coil pairs, namely a second coil pair 220′, a third coil pair 220″ and a fourth coil pair 220″′ which together are called drive means 220 in the following. For example, the second coil pair 220′ generates a component of the magnetic drive field 221 which extends in the direction of the coil axis of the first coil pair 210′, 210″ or the selection means 210, i.e. for example vertically. To this end the windings of the second coil pair 220′ are traversed by equal currents in the same direction. The effect that can be achieved by means of the second coil pair 220′ can in principle also be achieved by the superposition of currents in the same direction on the opposed, equal currents in the first coil pair 210′, 210″, so that the current decreases in one coil and increases in the other coil. However, and especially for the purpose of a signal interpretation with a higher signal to noise ratio, it may be advantageous when the temporally constant (or quasi constant) selection field 211 (also called gradient magnetic field) and the temporally variable vertical magnetic drive field are generated by separate coil pairs of the selection means 210 and of the drive means 220.

The two further coil pairs 220″, 220″′ are provided in order to generate components of the magnetic drive field 221 which extend in a different direction in space, e.g. horizontally in the longitudinal direction of the region of action 300 (or the patient 350) and in a direction perpendicular thereto. If third and fourth coil pairs 220″, 220′ of the Helmholtz type (like the coil pairs for the selection means 210 and the drive means 220) were used for this purpose, these coil pairs would have to be arranged to the left and the right of the region of treatment or in front of and behind this region, respectively. This would affect the accessibility of the region of action 300 or the region of treatment 300. Therefore, the third and/or fourth magnetic coil pairs or coils 220″, 220″′ are also arranged above and below the region of action 300 and, therefore, their winding configuration must be different from that of the second coil pair 220′. Coils of this kind, however, are known from the field of magnetic resonance apparatus with open magnets (open MRI) in which an radio frequency (RF) coil pair is situated above and below the region of treatment, said RF coil pair being capable of generating a horizontal, temporally variable magnetic field. Therefore, the construction of such coils need not be further elaborated herein.

The arrangement 10 according to the present invention further comprise receiving means 230 that are only schematically shown in FIG. 1. The receiving means 230 usually comprise coils that are able to detect the signals induced by magnetization pattern of the magnetic particles 100 in the region of action 300. Coils of this kind, however, are known from the field of magnetic resonance apparatus in which e.g. a radio frequency (RF) coil pair is situated around the region of action 300 in order to have a signal to noise ratio as high as possible. Therefore, the construction of such coils need not be further elaborated herein.

In an alternative embodiment for the selection means 210 shown in FIG. 1, permanent magnets (not shown) can be used to generate the gradient magnetic selection field 211. In the space between two poles of such (opposing) permanent magnets (not shown) there is formed a magnetic field which is similar to that of FIG. 2, that is, when the opposing poles have the same polarity. In another alternative embodiment of the arrangement according to the present invention, the selection means 210 comprise both at least one permanent magnet and at least one coil 210′, 210″ as depicted in FIG. 2.

The frequency ranges usually used for or in the different components of the selection means 210, drive means 220 and receiving means 230 are roughly as follows: The magnetic field generated by the selection means 210 does either not vary at all over the time or the variation is comparably slow, preferably between approximately 1 Hz and approximately 100 Hz. The magnetic field generated by the drive means 220 varies preferably between approximately 25 kHz and approximately 100 kHz. The magnetic field variations that the receiving means are supposed to be sensitive are preferably in a frequency range of approximately 50 kHz to approximately 10 MHz.

FIGS. 4a and 4b show the magnetization characteristic, that is, the variation of the magnetization M of a particle 100 (not shown in FIGS. 4a and 4b) as a function of the field strength H at the location of that particle 100, in a dispersion with such particles. It appears that the magnetization M no longer changes beyond a field strength +H_{c} and below a field strength −H_{c}, which means that a saturated magnetization is reached. The magnetization M is not saturated between the values +H_{c} and −H_{c}.

FIG. 4a illustrates the effect of a sinusoidal magnetic field H(t) at the location of the particle 100 where the absolute values of the resulting sinusoidal magnetic field H(t) (i.e. “seen by the particle 100”) are lower than the magnetic field strength required to magnetically saturate the particle 100, i.e. in the case where no further magnetic field is active. The magnetization of the particle 100 or particles 100 for this condition reciprocates between its saturation values at the rhythm of the frequency of the magnetic field H(t). The resultant variation in time of the magnetization is denoted by the reference M(t) on the right hand side of FIG. 4a. It appears that the magnetization also changes periodically and that the magnetization of such a particle is periodically reversed.

The dashed part of the line at the centre of the curve denotes the approximate mean variation of the magnetization M(t) as a function of the field strength of the sinusoidal magnetic field H(t). As a deviation from this centre line, the magnetization extends slightly to the right when the magnetic field H increases from −H_{c} to +H_{c} and slightly to the left when the magnetic field H decreases from +H_{c} to −H_{c}. This known effect is called a hysteresis effect which underlies a mechanism for the generation of heat. The hysteresis surface area which is formed between the paths of the curve and whose shape and size are dependent on the material, is a measure for the generation of heat upon variation of the magnetization.

FIG. 4b shows the effect of a sinusoidal magnetic field H(t) on which a static magnetic field H₁ is superposed. Because the magnetization is in the saturated state, it is practically not influenced by the sinusoidal magnetic field H(t). The magnetization M(t) remains constant in time at this area. Consequently, the magnetic field H(t) does not cause a change of the state of the magnetization.

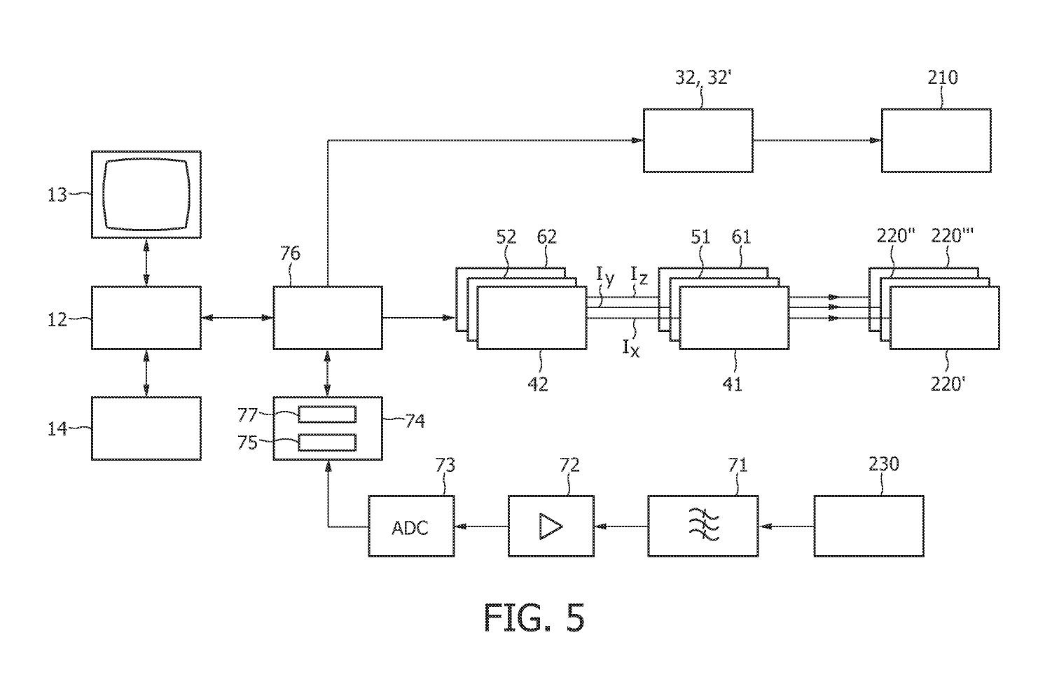

FIG. 5 shows a block diagram of the apparatus 10 shown in FIG. 1. The selection means 210 is shown schematically in FIG. 5. Preferably, the selection means 210 are provided with three magnetic selection field generation means, in particular either coils, permanent magnets or a combination of coils and permanent magnets. Said three magnetic selection field generation means are preferably arranged such that for each spatial direction one magnetic selection field generation means is provided. If in an embodiment coil pairs are provided as magnetic selection field generation means, the coil pairs are supplied with a DC current from a controllable current source 32, said current source 32 being controlled by the control means 76. In order to individually set the gradient strength of the selection field 211 in a desired direction, an overlaid current is overlaid to at least one of coil pairs, wherein the overlaid current of opposed coils is oppositely oriented. In a preferred embodiment, the control means 76 furthermore controls that the sum of the field strength and the sum of the gradient strength of all three spatial fractions of the selection field 211 is maintained at a predefined level.

If in an embodiment permanent magnets are provided as magnetic selection field generation means instead of coil pairs, the current source 32 need to be exchanged by an actuation means 32′, e.g. an electro motor, which is able to mechanically move the permanent magnets in order to set the gradient strength in the desired direction according to the control signals provided by the control means 76.

The control means 76 is in turn connected to a computer 12 which is coupled to a monitor 13 for displaying the distribution of magnetic particles in the examination area and an input unit 14, for example a keyboard. A user is therefore able to set the desired direction of the highest resolution and in turn receives the respective image of the region of action on the monitor 13. If the critical direction, in which the highest resolution is needed, deviates from the direction set first by the user, the user can still vary the direction manually in order to produce a further image with an improved imaging resolution. This resolution improvement process can also be operated automatically by the control means 76 and the computer 12. The control means 76 in this embodiment sets the gradient field in a first direction which is automatically estimated or set as start value by the user. The direction of the gradient field is then varied stepwise until the resolution of the thereby received images, which are compared by the computer 12, is maximal, respectively not improved anymore. The most critical direction can therefore be found respectively adapted automatically in order to receive the highest possible resolution.

The coil pairs (second magnetic means) 220′, 220″, 220′″ are connected to current amplifiers 41, 51, 61, from which they receive their currents. The current amplifiers 41, 51, 61 are in turn in each case connected to an AC current source 42, 52, 62 which defines the temporal course of the currents Ix, Iy, Iz to be amplified. The AC current sources 42, 52, 62 are controlled by the control means 76.

The receiving coil (receiving means) is also shown schematically in FIG. 5. The signals induced in the receiving coil 230 are fed to a filter unit 71, by means of which the signals are filtered. The aim of this filtering is to separate measured values, which are caused by the magnetization in the examination area which is influenced by the change in position of the two part-regions (301, 302), from other, interfering signals. To this end, the filter unit 71 may be designed for example such that signals which have temporal frequencies that are smaller than the temporal frequencies with which the coil pairs 220′, 220″, 220″′ are operated, or smaller than twice these temporal frequencies, do not pass the filter unit 71. The signals are then transmitted via an amplifier unit 72 to an analog/digital converter 73 (ADC). The digitalized signals produced by the analog/digital converter 73 are fed to an image processing unit (also called reconstruction means) 74, which reconstructs the spatial distribution of the magnetic particles from these signals and the respective position which the first part-region 301 of the first magnetic field in the examination area assumed during receipt of the respective signal and which the image processing unit 74 obtains from the control means 76. The reconstructed spatial distribution of the magnetic particles is finally transmitted via the control means 76 to the computer 12, which displays it on the monitor 13.

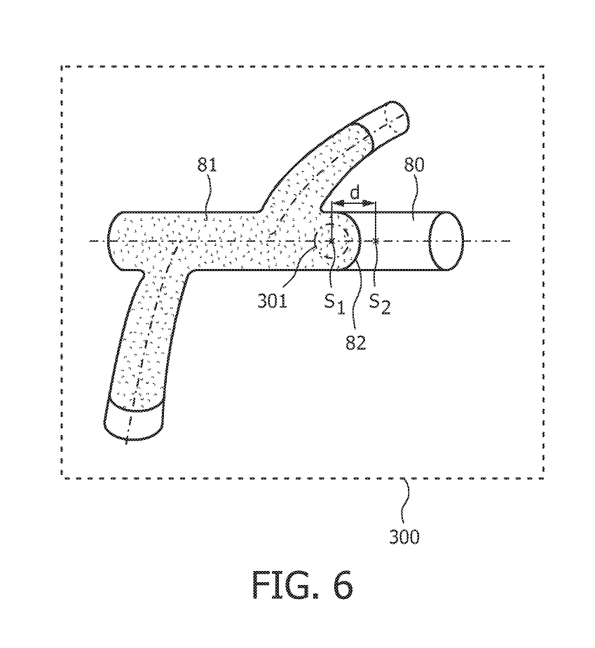

According to the present invention the control means 76 is adapted for controlling the drive means, i.e. the coil pairs 220′, 220″, 220′″, to change the position in space of the first sub-zone 301, i.e. the FPP, so that it follows a vessel 80 (see FIG. 6), which is located within the region of action 300 and within which a liquid containing a magnetic material flows. For instance, the vessel 80 can be a coronary artery within which blood is flowing whose velocity shall be measured. The control means 76 is further adapted for controlling the receiving means, i.e. the receiving coil 230, to acquire at least two detection signals at different positions S₁, S₂ of the FFP 301 along the vessel 80, i.e. the FFP is moved (once or several times) to positions S₁, S₂ and each time a detection signal is acquired. The fluid in the vessel portion located in the region of action 300 (e.g. the blood in the artery) contains a contrast agent 81 containing the above described magnetic material which is response to MPI signal excitation. Preferably, the detection signals are acquired while a bolus 82 of said contrast agent 81 is passing said positions S₁, S₂. In particular, fluctuations of the bolus are detected at said positions.

The processing unit 74 comprises a correlation unit 75 for correlating two of the detections signals. The correlation unit 75 could, for instance, be adapted such that it compares two detections signals and identifies characteristic points in the detections signals from which the time delay Δt is calculated after which the same signal acquired at the first position S₁ has been acquired at the second position S₂. From the correlated detection signal and the known distance d between the positions S₁, S₂ of the FFP, at which said detections signals were acquired, as well as the calculated time delay Δt between said measurements the local velocity v of the liquid (v=d/Δt) is then determined.

In a particular embodiment the correlation unit 75 is adapted to determine Δt =t′-t (t′ being the time of measurement at S₂, t being the time of measurement at S₁) such that the convolution ∫S₁(t′)S₂(t′−t)dt′ is maximized. To solve this known means, e.g. a fourier transform, can be applied.

Preferably, a plurality of detections signals are acquired at said two (or more) positions S₁, S₂ along the vessel 80, and different two detection signals of the plurality of detections signals are correlated (preferably, a plurality of pairs of detections signals are correlated). From the correlated detection signal and the known distance between the positions, at which said detections signals were acquired, the local velocity of the liquid at different positions is the determined a couple of times (corresponding to the number of pairs of detection signals used for the correlation), preferably for all detection signals of voxel pairs with short enough distance.

For following the vessel during the acquisition of the detection signals a roadmap of the vessel 80 (or the complete vessel tree in the region of action) is used in an embodiment. This vessel tree can be obtained in advance, e.g. by a different modality (e.g. CT or MR), or it can be acquired in a first step by MPI and by use of a first bolus of a contrast agent. In the latter case a segmentation 77 for segmenting vessels 80 from acquired detection signals to obtain said roadmap is provided in the processing unit 74.

For changing the position in space of the region of action 300 by means of a magnetic focus field a focus means (not shown) can be provided. This focus means can be adapted such that the FFP is following the vessels with high velocity. Typically, separate coils are used as focus means, but it is also possible to use the same coils as used for the drive means.

According to an embodiment the FFP is moved along the vessel(s) of interest, in particular by use of the vessel tree. For this purpose homogenous magnetic fields are used with large amplitude (i.e. focus fields) and low speed (e.g. 10 m/s). Superposed to these focus fields are fast moving fields (i.e. drive fields with e.g. 1000 m/s).

For physiologic reasons the amplitude of the drive fields can often not be selected large enough to move the FFP completely along a vessel of interest (e.g. the coronary arteries). Hence, it is preferred to move the FFP back and forth along the same segment of the vessel and to measure detections signals from the same segment a couple of times.

According to the present invention images can be reconstructed in real time. For instance, an image can be reconstructed on of which the calculated velocities can be displayed, e.g. color coded.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality. A single element or other unit may fulfill the functions of several items recited in the claims. The mere fact that certain measures are recited in mutually different dependent claims does not indicate that a combination of these measured cannot be used to advantage.

A computer program may be stored/distributed on a suitable medium, such as an optical storage medium or a solid-state medium supplied together with or as part of other hardware, but may also be distributed in other forms, such as via the Internet or other wired or wireless telecommunication systems.

Any reference signs in the claims should not be construed as limiting the scope.

1. An arrangement (10) for measuring the local velocity of a liquid containing a magnetic material (100) in a vessel (80) within a region of action (300), which arrangement comprises:
selection means (210) for generating a magnetic selection field (211) having a pattern in space of its magnetic field strength such that a first sub-zone (301) having a low magnetic field strength and a second sub-zone (302) having a higher magnetic field strength are formed in the region of action (300),
drive means (220) for changing the position in space of the two sub-zones (301, 302) in the region of action (300) by means of a magnetic drive field (221) so that the magnetization of the magnetic material (100) changes locally,
receiving means (230) for acquiring detection signals, which detection signals depend on the magnetization in the region of action (300), which magnetization is influenced by the change in the position in space of the first and second sub-zone (301, 302),
control means for controlling the drive means (220) to change the position in space of the first sub-zone (301) so that it follows the vessel and for controlling the receiving means (230) to acquire at least two detection signals at different positions (S₁, S₂) of the first sub-zone (301) along the vessel (80), and
correlation means (75) for correlating two of the at least two detections signals and for determining from the correlated detection signal and the known distance (d) between the positions (S₁, S₂) of the first sub-zone (301), at which said detections signals were acquired, the local velocity of the liquid.

2. An arrangement (10) as claimed in claim 1,
wherein said control means (76) is adapted for controlling the receiving means (230) to acquire at least two detection signals at different positions (S₁, S₂) of the first sub-zone (301) along the vessel (80) while a bolus (82) of a medium (81), in particular a contrast agent, containing said magnetic material (100) is passing said different positions (S₁, S₂).

3. An arrangement (10) as claimed in claim 1,
wherein said control means (76) is adapted for controlling the receiving means (230) to acquire a plurality of detection signals at different positions (S₁, S₂) of the first sub-zone (301) along the vessel (80) and said correlation means (75) is adapted for manifold correlating different two detection signals of the plurality of detections signals and for manifold determining from the correlated detection signal and the known distance between the positions (S₁, S₂) of the first sub-zone (301), at which said detections signals were acquired, the local velocity of the liquid at different positions (S₁, S₂) along the vessel (80).

4. An arrangement (10) as claimed in claim 1,
wherein said control means (76) is adapted for using a roadmap of the vessel (80) along which the at least two detection signals shall be acquired and for using said roadmap to control the drive means.

5. An arrangement (10) as claimed in claim 4,
wherein said control means (76) is adapted for controlling the drive means (220) and the receiving means (230) to acquire said roadmap.

6. An arrangement (10) as claimed in claim 5,
wherein said control means (76) is adapted for controlling the drive means (220) and the receiving means (230) to acquire detection signals at different positions (S₁, S₂) of the first sub-zone (301) in said region of action (300) while a bolus (82) of a medium (81), in particular a contrast agent, containing said magnetic material (100) is passing said different positions (S₁, S₂), and
further comprising a segmentation means (77) for segmenting vessels (80) from said acquired detection signals to obtain said roadmap.

7. An arrangement (10) as claimed in claim 6,
further comprising focus means for changing the position in space of the region of action (300) by means of a magnetic focus field wherein control means is adapted for controlling the focus means to move the region of action for the acquisition of said roadmap.

8. An arrangement (10) as claimed in claim 6,
further comprising reconstruction means (74) for reconstructing a concentration image of said region of action from said detections signal acquired at different positions of the first sub-zone (301) in said region of action.

9. An arrangement (10) as claimed in claim 1,
wherein said control means (76) is adapted for controlling the drive means (220) and/or the focus means to change the position in space of the first sub-zone (301) such that it substantially moves in a direction along the expected movement of the liquid in the vessel (80).

10. An arrangement (10) as claimed in claim 1,
wherein said control means (76) is adapted for controlling the drive means (220) and/or the focus means to change the position in space of the first sub-zone (301) such that it substantially moves on a surface of a known object.

11. A method for measuring the local velocity of a liquid containing a magnetic material (100) in a vessel (80) within a region of action (300), which method comprises the steps of:
generating a magnetic selection field (211) having a pattern in space of its magnetic field strength such that a first sub-zone (301) having a low magnetic field strength and a second sub-zone (302) having a higher magnetic field strength are formed in the region of action (300),
changing the position in space of the two sub-zones (301, 302) in the region of action (300) by means of a magnetic drive field (221) so that the magnetization of the magnetic material (100) changes locally,
acquiring detection signals, which detection signals depend on the magnetization in the region of action (300), which magnetization is influenced by the change in the position in space of the first and second sub-zone (301, 302),
controlling the changing of the position in space of the first sub-zone (301) so that it follows the vessel,
acquiring at least two detection signals at different positions (S₁, S₂) of the first sub-zone (301) along the vessel (80),
correlating two of the at least two detections signals, and
determining from the correlated detection signal and the known distance (d) between the positions (S₁, S₂) of the first sub-zone (301), at which said detections signals were acquired, the local velocity of the liquid.

12. Computer program comprising program code means for causing a computer to control an arrangement as claimed in claim 1 to carry out the steps of the method as claimed in claim 11 when said computer program is carried out on the computer.
